(19) United States
(12) Patent Application Publication
(10) Pub. No.: US 2011/0264094 A1
(43) Pub. Date: Oct. 27, 2011

(54) EXTERNAL FIXATION CLAMP

(76) Inventors: Mark Richard Cunliffe, West Yorkshire (GB); Malcolm Graham Ness, Northumberland (GB); Karl Herbert Kraus, Atnes, IA (US)

(21) Appl. No.: 13/057,390

(22) PCT Filed: Aug. 4, 2009
(86) PCT No.: PCT/GB2009/001912

§ 371 Date: Jul. 6, 2011

(30) Foreign Application Priority Data
Aug. 5, 2008 (GB) 0814263.0

(51) Int. Cl. A61B 17/00

(52) U.S. Cl. 606/59

(57) ABSTRACT

An external fixation clamp for attaching a fixation pin to a connecting rod, the clamp including a clamp body having a slot dimensioned to transversely receive the connecting rod and a fixation pin bore adapted to receive the fixation pin. The clamp body further includes a set screw bore for receiving a set screw, the set screw bore intersecting the slot and the fixation pin bore. The clamp further includes a set screw adapted to be received in the set screw bore and to extend into the slot and the fixation pin bore so clamping the connecting rod and fixation pin in place.

### CROSS-REFERENCE TO RELATED APPLICATIONS

The subject patent application claims priority to and all the benefits of International Application No. PCT/GB2009/001912, which was filed on Aug. 4, 2009, with the World Intellectual Property Organization, and which claims priority to GB 0814263.0 filed Aug. 5, 2008.

### BACKGROUND OF THE INVENTION

The present invention relates to an external fixation clamp for attaching a fixation pin to a connecting rod. More particularly, but not exclusively, the present invention relates to an external fixation clamp comprising a clamp body, the clamp body having a slot to receive a connecting rod, a fixation pin bore to receive a fixation pin and a set screw bore for receiving a set screw, the set screw being adapted to fix the connecting rod and fixation pin in place on insertion into the set screw bore.

External fixation clamps are known. U.S. Pat. No. 5,976,133 discloses an external fixation clamp for attaching a fixation pin to a connecting rod. Such a clamp however is difficult to employ requiring a high degree of manual dexterity. Such a clamp also allows only specific diameters of fixation pins. Even slight variations in fixation pin diameters render the fixation clamp non-functional. The clamp and fixation pin must therefore be manufactured using very high manufacturing tolerances. This increases the cost of manufacture. U.S. Pat. No. 5,921,985 discloses an external fixation clamp for attaching a fixation pin to a connecting rod. This clamp is composed of six separate components, is difficult to assemble, and requires two wrenches to tighten.

### BRIEF SUMMARY OF THE INVENTION

The present invention seeks to overcome the problems of the prior art.

Accordingly, in a first aspect, the present invention provides an external fixation clamp for attaching a fixation pin to a connecting rod, the clamp including a clamp body having a slot dimensioned to transversely receive the connecting rod and a fixation pin bore adapted to receive the fixation pin. The clamp body further includes a set screw bore for receiving a set screw, the set screw bore intersecting the slot and the fixation pin bore. The clamp further includes a set screw adapted to be received in the set screw bore and to extend into the slot and the fixation pin bore so clamping the connecting rod and fixation pin in place.

The external fixation clamp according to the invention is simpler to use than existing external fixation clamps. By inserting the set screw into the set screw bore both the connecting rod and fixation pin can be clamped in position relative to the clamp body. The fixation clamp is composed of only two pieces, the clamp body and the set screw. No complex clamp assembly steps are required.

In addition, the external fixation clamp according to the invention can be manufactured with lower tolerances than existing clamps which reduces manufacturing cost.

Preferably, the set screw bore is arranged such that on insertion of the set screw into the set screw bore the set screw first extends into the slot and then on further insertion extends into the fixation pin bore.

At least one of the side walls of the slot can be textured.

Preferably, the slot is adapted to receive a connecting rod by a snap fit.

The external fixation clamp can further comprise a connecting rod.

The connecting rod can have a non-linear stiffness, and is preferably carbon fibre.

Preferably, the set screw is adapted to urge the connecting rod against a side wall of the slot to clamp the connecting rod in place.

The thread on the set screw can be adapted to cut a thread in the outer face of the connecting rod.

The set screw can be adapted to urge the fixation pin against the fixation pin bore to clamp the fixation pin in place.

The external fixation clamp can further comprise a fixation pin within the fixation pin bore.

The fixation pin bore can be non-circular, and preferably square or rectangular.

The fixation pin bore can comprise a V groove for receiving the fixation pin.

### BRIEF DESCRIPTION OF THE SEVERAL VIEWS OF THE DRAWINGS

The present invention will now be described by way of example only and not in any limitative sense with reference to the accompanying diagrams in which:

FIG. 1 shows a known assembled external fixation clamp in perspective view;

FIG. 2 shows the clamp body of an external fixation clamp according to the invention in perspective view;

FIG. 3 shows an external fixation clamp according to the invention in combination with a connecting rod and fixation pin;

FIGS. 4(a) to 4(c) shown insertion of a fixation pin into an external fixation clamp according to the invention;

FIG. 5 shows an external fixation clamp according to the invention from above;

FIGS. 6(a) and 6(b) show a further embodiment of an external fixation clamp according to the invention in side and end views; and,

FIGS. 7(a) and 7(b) show a further embodiment of an external fixation clamp according to the invention in side and end views.

### DETAILED DESCRIPTION OF THE EXEMPLARY EMBODIMENT(S)

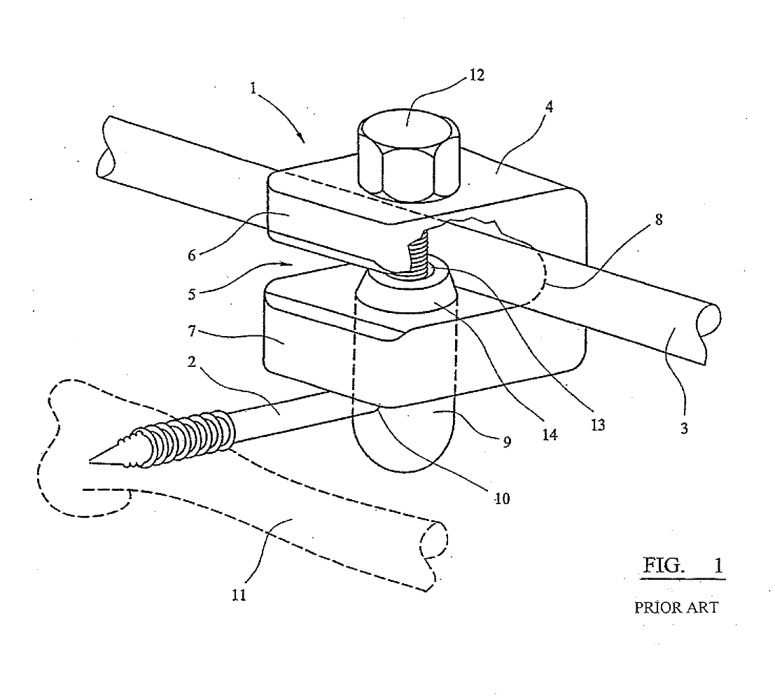

Shown in FIG. 1 is a known external fixation clamp 1 in combination with a fixation pin 2 and connecting rod 3 in perspective view. The clamp 1 comprises a clamp body 4 having a slot 5 dimensioned to receive the connecting rod 3. Connector bores (not shown) extend through the two side walls 6,7 of the slot 5.

In use a connecting rod 3 is inserted into the slot 5 until it abuts the base 8 of the slot 5. A pin connector 9 is then provided. The pin connector 9 has a bore 10 extending there through perpendicular to the length of the pin connector 9. The bore 10 of the pin connector 9 is slid onto a fixation pin 2 extending from the bone 11 of a patient. Once the pin connector 9 is the correct distance from the patient a connector bore of the clamp body 4 is slid onto the pin connector 9. A screw 12 is inserted into the opposite connector bore and threaded into engagement with a corresponding recess 13 in the pin connector 9. Rotation of the screw 12 draws the pin connector 9 into its connector bore until a beveled surface 14 on the pin connector 9 abuts the connecting rod 3, fixing the rod in place in the slot 5. The bore 10 in the pin connector 9 is arranged such that as the beveled surface 14 abuts the connecting rod 3 the fixation pin 2 abuts the clamp body 4, fixing the fixation pin 2 in position.

Assembly of this known clamp 1 requires a high degree of manual dexterity. This is not desirable in a surgical procedure. In addition, in order for the clamp 1 to work effectively the fixation pin 2 must abut the outer wall of the clamp body 4 exactly as the beveled surface 14 abuts the connecting rod 3. The clamp 1 must therefore be manufactured with a very high degree of precision, increasing manufacturing costs.

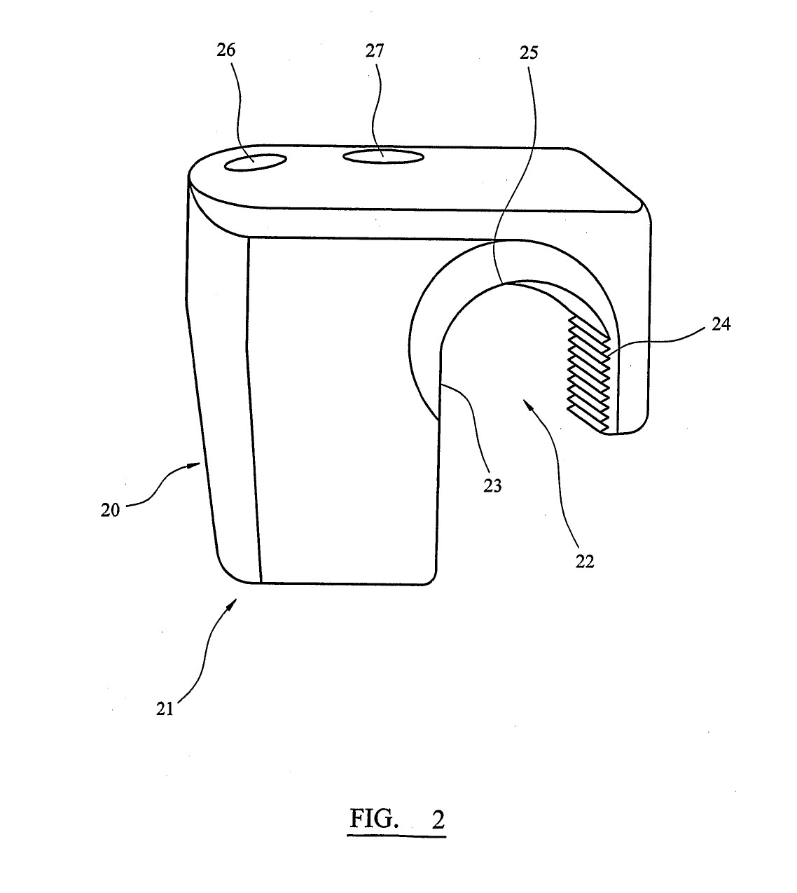

Shown in FIG. 2 is a clamp body 20 of an external fixation clamp 21 according to the invention in perspective view. The clamp body 20 comprises a slot 22. The slot 22 is dimensioned to transversely receive a connecting rod (not shown). One of the two sides 23,24 of the slot 22 is textured as shown. The base 25 of the slot 22 is smooth.

Extending through the clamp body 20 is a fixation pin bore 26 having a circular cross section and dimensioned to receive a fixation pin (not shown). The clamp body 20 also comprises a set screw bore 27 for receiving a set screw (not shown). In this embodiment the set screw bore 27 lies in the same plane as the fixation pin bore 26 and the slot 22 and is inclined to the fixation pin bore 26. The set screw bore 27 intersects both the slot 22 and the fixation pin bore 26.

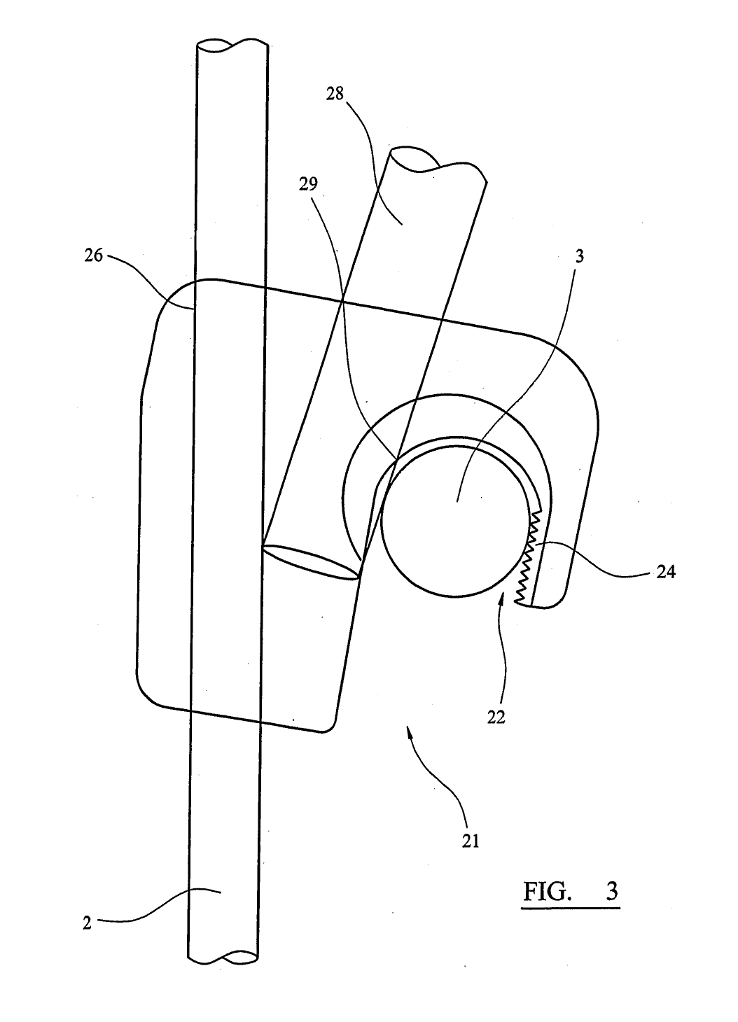

FIG. 3 shows an external fixation clamp 21 according to the invention in an assembled state, clamping a connecting rod 3 and fixation pin 2 in place. In use a connecting rod 3 is positioned within the slot 22. In this embodiment the connecting rod 3 snap fits within the slot 22.

A fine pitch high hardness set screw 28 is then threaded into engagement with a corresponding threaded surface (not shown) on the inner face of the set screw bore 27. As the set screw 28 is turned about its axis it slowly enters into the set screw bore 27. When the set screw 28 reaches the point 29 where the set screw bore 27 intersects with the slot 22, the outer face of the set screw 28 abuts against the connecting rod 3 urging the connecting rod 3 against the side 24 of the slot 22, clamping the connecting rod 3 in place. The outer face of the set screw 28 cuts threads in the outer face of the connecting rod 3 further preventing rotation of the connecting rod 3 in the slot 22.

Once the connecting rod 3 is clamped in place in the clamp body 20, the fixation pin bore 26 is slid along a fixation pin 2 extending from the bone of a patient until the clamp body 20 is correctly positioned relative to the patient. The set screw 28 is then turned further until the end of the set screw 28 abuts the fixation pin 2. Further turning of the set screw 28 urges the fixation pin 2 against the fixation pin bore 26 so preventing further movement of the fixation pin 2 within the fixation pin bore 26, clamping it in place.

The external fixation clamp 21 according to the invention can be assembled without difficulty. It is only required to turn the set screw 28 to clamp both the connecting rod 3 and fixation pin 2 in place. In addition, a high degree of precision in the dimensions of the clamp 21 according to the invention is not required, so reducing manufacturing cost. As the set screw 28 engages the connecting rod 3 it is displaced slightly towards the fixation pin 2. As the set screw 28 starts to engage the fixation pin 2 it is displaced slightly towards the connecting rod 3. The more the set screw 28 is inserted the more it pushes in both directions. The external fixation clamp 1 according to the invention can therefore accommodate any number of different fixation pin sizes through a range. In addition, this removes problems with manufacturing flaws, differences between English and Metric fixation pins and the products of different manufacturers. In addition, the external fixation clamp 21 can allow for variations in the locations of the set screw bore 27, fixation pin bore 26 and width of the slot 22, and still effectively clamp both the connecting rod 3 and fixation pin 2. The inherent wobble of the set screw 28 within the threads of the set screw bore 27 of the external fixation clamp 21 are eliminated because as the set screw 28 engages the fixation pin 2 at an angle, the fixation pin 2 pushes the set screw 28 against the connecting rod 3.

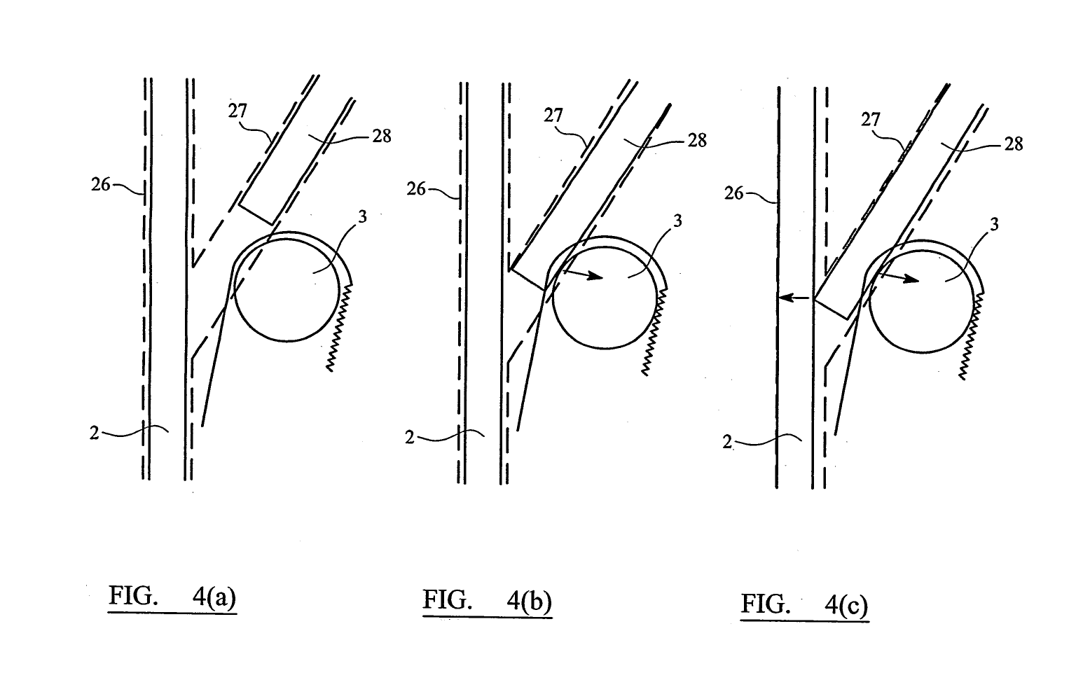

Entry of the set screw 28 into the external fixation clamp 21 is shown schematically in more detail in FIGS. 4(a) to 4(c). In a first step the set screw 28 is screwed into engagement with the set screw bore 27 (FIG. 4(a)). As the set screw 28 is screwed further into the set screw bore 27, the set screw 28 urges the connecting rod 3 against the wall of the slot 2, fixing the connecting rod 3 in place (FIG. 4(b)). As the set screw 28 is further screwed into the set screw bore 27 it abuts against the fixation pin 2, pushing the fixation pin 2 towards the side wall of the fixation pin bore 26. When the fixation pin 2 reaches the side wall of the fixation pin bore 26, further rotation of the set screw 28 firmly fixes the set screw 28 in position between the connecting rod 3 and fixation pin 2 (FIG. 4(c)). This eliminates the inherent wobble of the set screw 28 in the set screw bore 27.

In the above embodiments the fixation pin bore 26 is circular in cross section. In alternative embodiments the fixation pin bore 26 is other than circular. The fixation pin bore 26 can for example be square or rectangular. The fixation pin 2 will have a corresponding cross section. Such non circular cross sections resist rotation of the clamp body 20 around the fixation pin 2. In alternative embodiments the fixation pin bore 26 can include a V groove, typically opposite the point of entry of the set screw 28 into the fixation pin bore 26. In use the fixation pin 2 is urged into the V groove so again resisting rotation of the clamp body 20 about the fixation pin 2. In further alternative embodiments the fixation pin bore 26 may be both non-circular and include at least one V groove. In one embodiment the cross section of the fixation pin bore 26 is substantially rectangular with three sides of the bore although one of the sides of the rectangle comprises two inclined faces together forming a V groove.

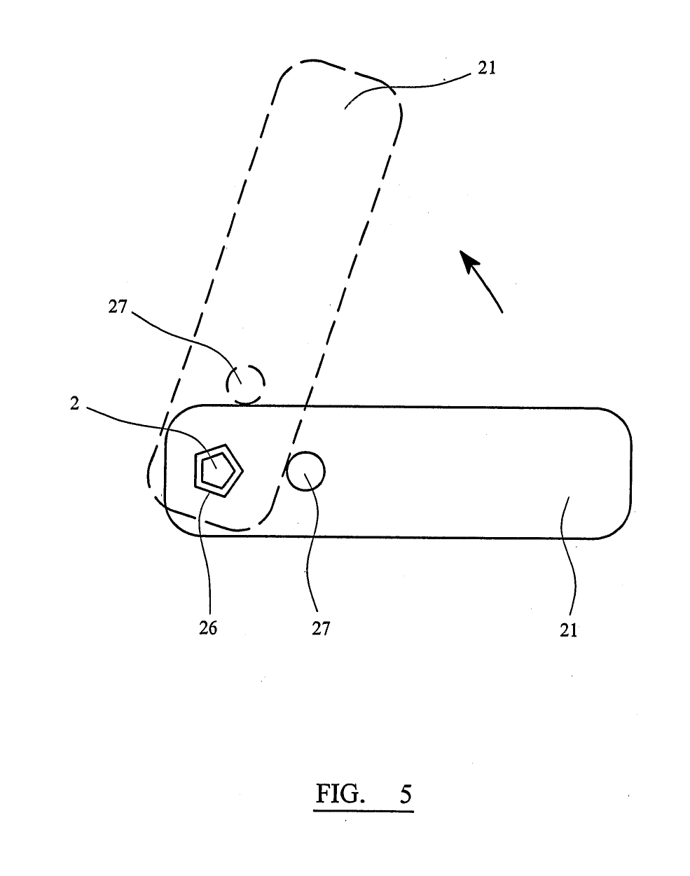

Shown in FIG. 5 is an external fixation clamp 21 according to the invention viewed from above. In this embodiment the fixation pin bore 26 is pentagonal in cross section. The fixation pin 2 is also pentagonal in cross section as shown. The external fixation clamp 21 can therefore be slid onto the fixation pin 2 in a variety of orientations as shown. This can be useful when aligning the external fixation clamp 21 with other fixation clamps (not shown).

In the above embodiment the fixation pin 2 is a complementary shape to the fixation pin bore 26, fitting closely into the fixation pin bore 26. The external fixation clamp 21 can therefore be fixed in a number of distinct positions with respect to the fixation pin 2. In each distinct position the fixation pin fits in the grooves in one distinct orientation. In an alternative embodiment the clamp 21 can be used with a fixation pin 2 which is non-complementary to the fixation pin bore 26 and in which the pin 2 fits into the one or more V grooves in a continuous range of orientations. The fixation pin 2 can for example be circular. In an alternative embodiment it can be elliptical. With such a non-complementary fixation pin 2 then as the clamp 21 is slid down the fixation pin 2 it can be rotated around the fixation pin 2 until the slot is parallel with the connecting rod 3. Once the connecting rod 3 is arranged within the slot the set screw 28 is tightened. The V shape of the fixation pin bore 26 receives the fixation pin 2, fixing it in place. The V shape will accommodate a variety of diameters of fixation pins 2. It will also support the fixation pin 2 along the length of the fixation pin bore 26. In all the above embodiments the set screw bore 27 lies in the same plane as the fixation pin bore 26 and slot 22, with the set screw bore 27 being inclined to the fixation pin bore 26. The inclination of the set screw bore 27 to the fixation pin bore 26 ensures that when the set screw 28 abuts the fixation pin 2 it mainly imparts a lateral force on the fixation pin 2, urging the pin 2 against the side wall of the fixation pin bore 26. Forces applied by the set screw 28 along the length of the fixation pin 2 cause ‘creep’ whereby the clamp body 20 moves along the fixation pin 2 as the set screw 28 is tightened. A large angle of inclination between the fixation pin bore 26 and the set screw bore 27 is therefore to be preferred to minimise this creep.

Creep of the connecting rod 3 is also a potential problem. As the set screw 28 extends into the slot 22 and abuts the connecting rod 3 the rotation of the set screw 28 may generate forces along the length of the connecting rod 3. This may cause the connecting rod 3 to creep through the slot 22. The roughened texture of the side wall(s) 23,24 of the slot 22 increases the coefficient of friction between the slot 22 and connecting rod 3 so reducing this creep.

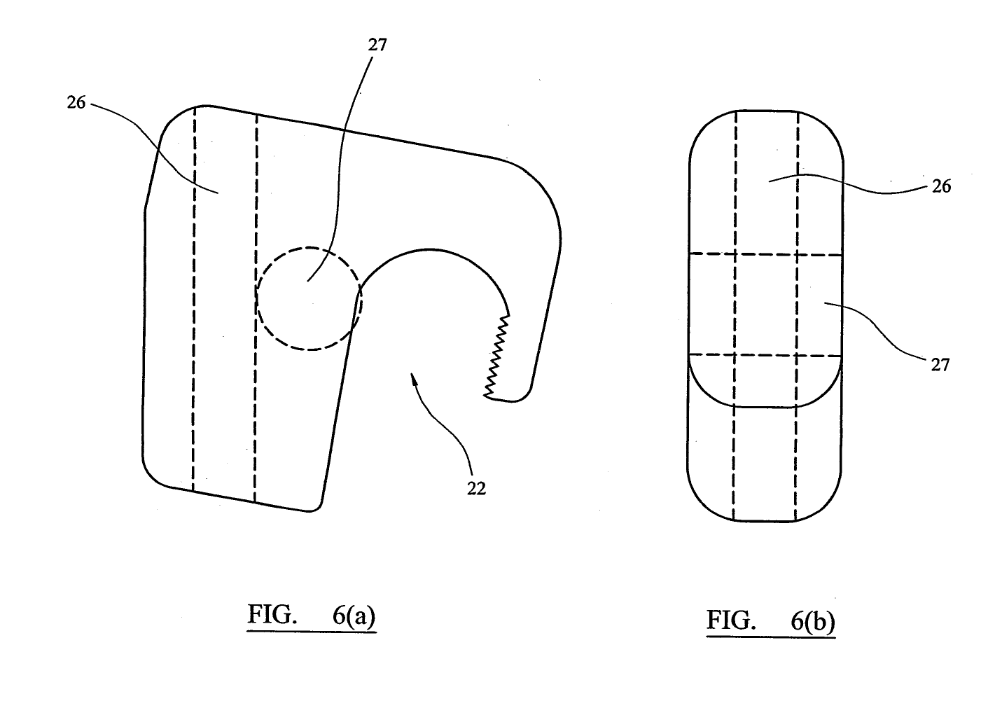

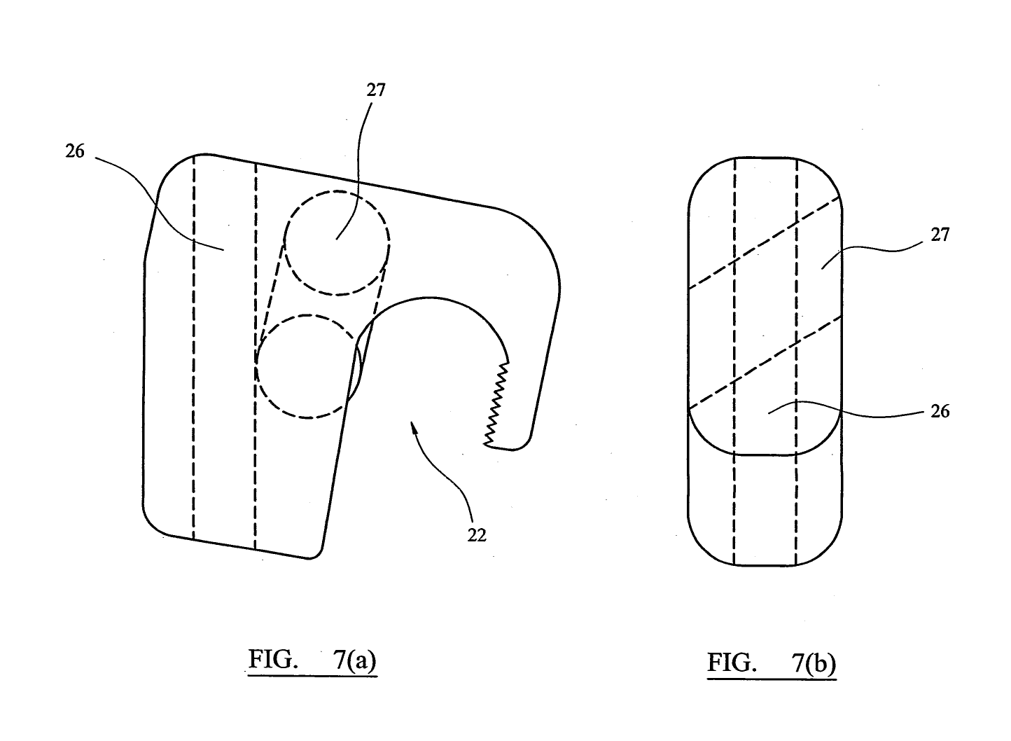

In alternative embodiments of the invention the fixation pin bore 26, slot 22 and set screw bore 27 are not coplanar. In one embodiment the set screw bore 27 is normal to the plane containing the fixation screw bore 26 and slot 22 as shown in side and end views in FIGS. 6(a) and 6(b). As the set screw 28 is inserted into the set screw bore 27 it simultaneously engages both the fixation pin 2 and connecting rod 3. This embodiment however suffers from creep along the connecting rod 3 as the direction of travel of the outer face of the set screw 28 is parallel to the length of the connecting rod 3. In a further embodiment of the invention the slot 22 lies in a different plane to the fixation pin bore 26 as shown in side and end views in FIGS. 7(a) and 7(b). The set screw bore 27 is inclined to both the slot 22 and the fixation pin bore 26 as shown. As the set screw 28 engages the connecting rod 3 the force on the connecting rod 3 is now inclined to the length of the connecting rod 3 so reducing creep.

The clamp body 20 is typically titanium as this is light, flexible and strong. The connecting rod 3 is typically a composite material with a non-linear stiffness or alternatively a light and strong material such as carbon fibre.

In embodiments of the invention where the set screw 28 cuts a thread in the connecting rod 3 the set screw 28 is typically made of a harder material than the connecting rod 3. In alternative embodiments the set screw 28 does not cut threads into the connecting rod 3 but simply urges the connecting rod 3 into abutment with the side wall 24 of the slot 22 to fix it in place.

1. An external fixation clamp for attaching a fixation pin to a connecting rod, the clamp comprising:
a clamp body having a slot dimensioned to transversely receive the connecting rod and a fixation pin bore adapted to receive the fixation pin;
the clamp body further having a set screw bore for receiving a set screw, the set screw bore intersecting the slot and the fixation pin bore;
the clamp further comprising a set screw adapted to be received in the set screw bore and to extend into the slot and the fixation pin bore so clamping the connecting rod and fixation pin in place relative to the clamp body.

2. An external fixation clamp as claimed in claim 1, wherein the set screw bore is arranged such that on insertion of the set screw into the set screw bore the set screw first extends into the slot and then on further insertion extends into the fixation pin bore.

3. An external fixation clamp as claimed in claim 1, wherein at least one of the side walls of the slot is textured.

4. An external fixation clamp as claimed in claim 1, wherein the slot is adapted to receive a connecting rod by a snap fit.

5. An external fixation clamp as claimed in claim 1, further comprising a connecting rod.

6. An external fixation clamp as claimed in claim 5, wherein the connecting rod has a non-linear stiffness.

7. An external fixation clamp as claimed in 1, wherein the set screw is adapted to urge the connecting rod against a side wall of the slot to clamp the connecting rod in place relative to the clamp body.

8. An external fixation clamp as claimed in claim 7, wherein the connecting rod has an outer face and the thread on the set screw is adapted to cut a thread in the outer face of the connecting rod.

9. An external fixation clamp as claimed in claim 1, wherein the set screw is adapted to urge the fixation pin against the fixation pin bore to clamp the fixation pin in place relative to the clamp body.

10. An external fixation clamp as claimed in claim 9, further comprising a fixation pin within the fixation pin bore.

11. An external fixation clamp as claimed in claim 1, wherein the fixation pin bore is non-circular.

12. An external fixation clamp as claimed in claim 1, wherein the fixation pin bore includes a V groove for receiving the fixation pin.

13. (canceled)

14. An external fixation clamp as claimed in claim 6, wherein the connecting rod is carbon fibre.

15. An external fixation clamp as claimed in claim 11, wherein the fixation pin bore is one of substantially square and substantially rectangular.
